(19) United States
(12) Patent Application Publication
(10) Pub. No.: US 2023/0320625 A1
(43) Pub. Date: Oct. 12, 2023

(54) Wearable Flexible Sensor Motion Capture System

(71) Applicants: Reuben Burch, Columbus, MS (US); Tony Luczak, Starkville, MS (US); David Saucier, Starkville, MS (US); John Ball, Starkville, MS (US); Harish Chander, Starkville, MS (US)

(72) Inventors: Reuben Burch, Columbus, MS (US); Tony Luczak, Starkville, MS (US); David Saucier, Starkville, MS (US); John Ball, Starkville, MS (US); Harish Chander, Starkville, MS (US)

(21) Appl. No.: 18/188,957

(22) Filed: Mar. 23, 2023

Related U.S. Application Data
(63) Continuation-in-part of application No. 16/506,932, filed on Jul. 9, 2019, now Pat. No. 11,672,480.
(60) Provisional application No. 62/695,602, filed on Jul. 9, 2018.

(51) Int. Cl. A61B 5/11; A61B 5/00; G01L 5/16

(52) U.S. Cl. CPC A61B 5/112; A61B 5/0004; A61B 5/4595; A61B 5/6807; A61B 5/7267; A61B 5/7275; G01L 5/16; A61B 2562/0261; A61B 2503/10; A61B 2505/09; A61B 2562/164

(57) ABSTRACT

The present design provides a novel system and device for wearables for humans and animals that capture and store kinematic and kinetic data and movement during training, rehabilitation, real-time events, and the like, analyze such data and movement in real-time during and after such activities, and provide output, feedback, assessment, and actionable biomechanical data and information about the wearer.

### CROSS-REFERENCE TO RELATED APPLICATIONS

This patent application is a Continuation In Part of U.S. Non-Provisional patent application Ser. No. 16/506,932, filed Jul. 9, 2019 by Reuben F. Burch V, et. al., and titled “Wearable Flexible Sensor Motion Capture System,” which claims the benefit of U.S. Provisional Patent Application No. 62/695,602, filed Jul. 9, 2018, which are hereby incorporated by reference in their entirety.

### STATEMENT OF GOVERNMENT SUPPORT

This invention was made with government support under grant numbers 1827652 and 1844451 awarded by the National Science Foundation. The government has certain rights in the invention.

### TECHNICAL FIELD

This disclosure relates to the field of kinesiology and physical therapy for humans and animals and, more specifically, to a novel system and device or apparatus for wearables or wearable materials for humans and animals that capture and store kinematic and kinetic data and movement during training, rehabilitation, real-time events, and the like, analyze such data, and provide output, feedback, and actionable information to and/or about the wearer.

### BACKGROUND

The present disclosure relates to a new system, device, and microprocessor-based software involving wearable devices for humans and animals that can capture, record, store, and analyze data and physical motion and movement parameter data during exercise, training, real-time events, sporting competitions, rehabilitation, and the like, and provide valuable feedback and information to the subject wearer and/or to medical or training personnel about the wearer. The disclosure provides a novel wearable flexible sensor motion capture and analysis system for assessing kinematic and kinetic motion and movement of an animal and/or human.

Biomechanical analyses of human joint range of motion (ROM) have evolved from simple goniometric measures to technologically-advanced optical motion capture systems. While motion capture technology aids in the assessment of joint range of motion with gold standard precision measures, the use of this technology is primarily confined within a laboratory setting, with limited applicability to changes in joint angles that occur in everyday tasks.

Traditionally, optical motion capture of biomechanical data collection is considered the gold standard for identifying kinematic and kinetic parameters and is generally confined to a research laboratory or lab-like environment due to the equipment requirements. Unfortunately, high costs and limited access to these research environments reduce the opportunity for improving all athletes, rehabilitation subjects, and the like through technical analysis. One promising technological advancement that has seen increased exposure in research, rehabilitation, and competition is wearable sensor technology and the opportunity to measure near real-time kinematics on the playing field and in subject assessment. Measuring various physiological and kinematic parameters is now accessible to the average athlete and test subject compared to the human and animal activity recognition devices from twenty years ago. Numerous commercially available products utilize micro electromechanical systems (MEMS), accelerometers, and gyroscopes to capture biomechanical measures outside the lab.

One of the benefits of using MEMS devices is that they offer a lower-cost alternative to traditional motion capture solutions. Using an inertial frame, the relative orientation of limb segments can be calculated from accelerometer and gyroscope data. One commonly-used type of MEMS is the inertial measurement unit, or IMU, and this type of sensor is found in most technologies where some form of movement information is captured. However, several recurring issues can be seen in IMU-based motion capture systems including distortion and drift and challenges in how to consistently manage calibration. The distortion and drift that affects actual sensor horizontal and vertical data are due to distortions in non-homogeneous magnetic fields, often caused by building construction materials and magnetic interference. To reduce noise, improved anatomical models and static calibration in defined positions have been developed. However, measurement errors still occur due to skin and segment speed of movement and axial segment rotation. According to Kavanagh et al., the separation of limb segment resultant acceleration could not be identified within the sensor data, resulting in the difficulty to obtain accurate measurements. Additionally, external devices are often incompatible with activities that involve contact and may require frequent adjustment and re-calibration, making them impractical for use in real-world environments.

In human movement, the neuromuscular system senses strains, positioning, and stretching of its proprioceptors and muscular system in order to coordinate limb segment movement and thereby joint movement. A body network sensor system that is capable of detecting such strain and stretch around the limb segments and thereby joint movements may offer an alternative to using stiff, circuit board-based IMUs in capturing human limb movement. Given calibration and consistency challenges that exist with IMUs used in the athletic wearable market today, a potential solution may lie in the use of a different kind of sensor, or sensors developed for a different purpose, such as soft robotic sensors. Totaro et al. custom designed soft sensors and integrated them into garments for precise movement validated in lower limb joints, but this research does not utilize “off-the-shelf” sensors and therefore are limited for future, real-world environment use cases. Other recent studies utilized more commercialized soft robot sensor solutions found in exoskeletons technologies for less complex movements not located around the foot and ankle. The present disclosure utilizes sensors, such as soft robotic sensors, that can be identified as silicone-textile (or other soft materials) layered with liquid conductive material and generally identified as resistive, capacitive, or inductive, or a combination thereof. As these sensors are stretched, their resistive, capacitive, or inductive values increase. At the beginning of the research that formed the basis of the disclosure, two primary soft robotic sensor solutions were available to test. Liquid Wire is a resistance-based sensor produced by Liquid Wire, Inc. and StretchSense™ is a capacitive-based sensor, both of which provide increased output values when stretched past their initial resting lengths in a linear fashion. Several advantages for using soft robotic sensors such as these include: (a) the ability to measure biomechanical strain without worry for occlusion errors that typically occur in optical systems and eliminate drift that can occur in MEMS sensors; (b) the realization of small changes in electromechanical specifications during loading and unloading; and (c) the reduction of interference as observed by the wearer. In addition, soft robotic sensors inherently offer “stretchability”, which allows the sensors to cover arbitrarily-shaped joints that occur on the human and animal body.

Wearable sensor technology incorporated into socks and other types of clothing exists. For example, U.S. Pat. No. 8,925,392, entitled “Sensors, Interfaces and Sensor Systems for Data Collection and Integrated Remote Monitoring of Conditions at or Near Body Surfaces”, discloses a sock that incorporates flexible and stretchable fabric-based pressure sensors. The device may be used for medical purposes like treatment of peripheral neuropathy and it has application in athletics to measure pressure on an athlete's lower extremities.

U.S. Pat. No. 9,427,179 discloses a sock or other garment with pressure sensors for measuring pressure or forces in feet, the stumps of limbs of an amputee fitted with prosthetic devises, or other parts of the body that are subject to pressure-inducing forces.

Products exist that involve sensors in socks that measure a runner's steps, speed, cadence, foot landing, and other measurements. Other patents are directed to wearable sensors that infer joint movement by placing sensors on different limb segments. For example, wearable joint action sensors are described in U.S. patent application Ser. No. 14/963,136. The device of that disclosure measures or detects joint movement by detecting the amount of separation between one limb and a proximity sensor attached to another limb.

Another device is disclosed in U.S. Pat. No. 8,961,439 entitled “System and Method for Analyzing Gait Using Fabric Sensors”. That device uses a tension or pressure sensor to sense or quantify a wearer's movement and calculates the wearer's gait by comparing the sensor's measurements with a set of gait parameters.

Due to the intricacy of the ankle complex, for example, precise placement of sensors are required to obtain accurate kinematic data during movement. Ankle complex rotational components can be found within the talocrural, subtalar, and inferior tibofibular joints. Given the anatomical design of the ankle joints, movement of the foot during open kinetic chain in plantar flexion and dorsiflexion do not occur in a single sagittal plane. During plantar flexion, the foot moves 28 degrees in the sagittal plane, one degree in the transverse plane, and four degrees in the frontal plane. Likewise, during dorsiflexion, there are 23 degrees of movement in the sagittal plane, nine degrees in the transverse plane, and two degrees in the frontal plane.

### SUMMARY

Unlike previous research on comparisons of IMUs for optimal motion capture which both ignore internal and external rotations and inversion and eversion, the present disclosure encompasses the viability of using soft robotic sensors to capture all movement in all three planes.

An important aspect of the disclosure is the consideration for placement of these sensors in order to optimize measurements of complex ankle and body part movements. Previous work by Mengüç et al. has evaluated the sensor placement at the posterior part of the ankle and heel, extending from the distal aspect of the gastrocnemius muscle complex down to the calcaneous, which has shown positive results in sagittal plane movements (coefficient of determination 0.9680). To capture tri-planar ankle joint movement, one sensor was placed parallel to the distal ⅓ aspect of the fibula, overlaying the lateral malleolus to capture inversion and eversion. A second sensor was placed vertically in-line with the distal ⅓ aspect of the tibia onto the superior aspect of the talus, and a third sensor was positioned perpendicular to the 23-degree axis of inversion. This novel analysis forms the basis of the disclosure provides a starting point for where sensors should be placed in order to effectively capture full range of ankle, and other joint, motion.

Subject matter experts (SMEs) have identified two additional wearable gaps: (a) there exists a lack of trust and confidence in data output and consistency of motion and movement tracking wearables currently available on the market; and (b) student and professional athletes are often noncompliant and resistant to using wearable technology due to the awkwardness of placement and general discomfort. The present disclosure addresses these issues and the trust and wearable comfort requirements identified by SMEs via complete transparency into data capture and calculations by publishing algorithms and research results and by integrating the system and apparatus or device of the present disclosure into existing wearable materials and into uniform requirements, for example.

The present disclosure and wearable technology provides a distinctive and novel system and device not found in existing technology or products. The disclosure discloses novel sensor placement and movement measurement. Moreover, soft robotic sensors (SRS), absolute joint angle measurements, use of a puck, i.e., a data acquisition and transmission module, and other features and components of the present disclosure described herein provide a unique system and device for capturing and assessing accurate kinematic and kinetic motion and movement parameters.

The present disclosure provides a new system and apparatus for wearable devices for humans and animals that capture, store, and record kinematic and kinetic data and movement during events in real-time in order to analyze such data and physical movement for exercise, training, sporting competitions, and rehabilitation, while providing valuable output, feedback, and actionable information to the subject wearer and/or to medical or training personnel about the wearer and relevant biomechanical data.

The disclosure comprises a body part wearable integrating soft robotic sensors (SRSs) into a wearable or compression wearable to capture kinematic and kinetic data of motion or movement during any physical activity, including rigorous training, competition, rehabilitation, athletics, and/or typical task events in real-world environments. Absolute (not inferred) joint angle data can be captured and analyzed in real-time or near real-time using machine learning derived from joint motion modeling and movement and relevant parameter data and through the analysis of data collected in participant motion and movement trials. Output and feedback from the device of the disclosure provides actionable information to the wearer and/or analyst or trainer about the level of risk associated with ankle or joint movement and placement, the forces applied to the foot and ankle, or other body part, symmetry across both of the wearer's complementary joints or body parts, and additional biomechanical information such as gait, dynamic compound movements (such as jumping), and distance, for example.

SRSs cover a broad range of fabric/cloth/soft materials, for example, that are integrated and embedded with resistive, capacitive, and/or inductive material that is flexible and conductive, providing changes in the electrical properties when stretched or pressure is applied. SRSs utilized in the disclosure are adapted in a wearable solution capable of accurately capturing kinematic and kinetic data in real-time or near real-time at the individual joint level to provide a meaningful assessment of use, risk of injury, or rehabilitation, for example. Data can be captured and analyzed utilizing machine learning from modeling of body part movements and via the analysis of data collected in participant movement trials. Information from the system and device of the disclosure provides actionable data, either typically machine readable and/or audible, and may include haptic information or feedback, to the wearer and trainer or tester about relevant body part movement and placement, the force(s) or pressures being applied to the body part, symmetry across the wearer's body part(s), relevant biomechanical information, and the like. The disclosure captures current, consistent, and accurate data “from the ground up” for making health and safety decisions about a wearer's ankles, toes, knees, elbows, wrists, fingers, and other body parts in need of analysis and functional measurement.

The disclosure utilizes SRSs to relate sensor stretch with the SRS output, either resistance, capacitance, or inductance, or a combination thereof. SRS sensors were analyzed and found to have linear characteristics, and thus are suitable for linear machine learning movement modeling. The machine learning component of the system can utilize either single or multiple sensor inputs to estimate kinetic and kinematic parameters and does not require the sensor outputs to be linear.

To optimize the design, SRS sensor placement on the human body is crucial for accurate and precise measurements. Sensor placement studies were performed for ankle complex plantar flexion, dorsiflexion, inversion, and eversion movements.

The plantar flexion SRSs were mounted on the dorsal surface of the foot to measure the downward movement of the foot, such as when the toes are pointed towards the ground and the angle between the dorsal surface of the foot and the lower leg increases. The SRS positions for this movement were determined based on the hallux (big toe) and surface of the top of the foot. The SRS was first oriented towards the hallux, then over the middle of the foot, and lastly towards the 5th phalanx. The SRS oriented towards the hallux was found to be optimal.

The dorsiflexion SRSs were mounted on the heel of the foot to measure the upward movement of the foot towards the lower leg (angle between the top of the foot and lower leg increases). There was only one choice for the dorsiflexion sensor, because the anatomy on the posterior side of the foot, primarily the calcaneus (heel), only provides one location for placement and orientation configuration (POC) to accurately measure dorsiflexion.

The inversion SRSs were mounted on the lateral side of the ankle to measure the movement of the sole (bottom of the foot) towards the midline of the body. These SRS locations were centered around the lateral malleolus (bony landmark on the lateral side of the ankle). The SRSs were first positioned anterior to the lateral malleolus, near to the 5th phalanx, then directly over the lateral malleolus, and finally, posterior to the lateral malleolus, close to the heel of the foot. The second position, directly over the lateral malleolus, was selected.

The eversion SRSs were mounted on the medial side of the ankle to measure the movement of the sole away from the midline of the body. The eversion SRSs were determined similarly to the inversion POCs, except they were based on the medial malleolus (bony landmark on the medial of the ankle). The middle position was chosen as best.

Once ankle placement studies had been performed, the system was compared to a state-of-the-art 3D multi-camera motion capture system utilizing MotionMonitor™ (Innovative Sports Training, Inc., Chicago, IL, USA) software. The MotionMonitor™ system outputs plantar flexion, dorsiflexion, inversion and eversion estimates based on the system tracking multiple body markers using a system of cameras and 3D software. The disclosure also estimates these outputs, and the design showed very high linear model fits and very low residuals compared to the motion capture outputs.

With the foregoing and other objects, features, and advantages of the present disclosure that will become apparent hereinafter, the nature of the disclosure may be more clearly understood by reference to the following detailed description, preferred embodiments, and appended claims.

### BRIEF DESCRIPTION OF THE DRAWINGS

These drawings accompany the detailed description of the disclosure and are intended to illustrate further the design and its advantages. The drawings, which are incorporated in and form a portion of the specification, illustrate certain preferred embodiments and, together with the entire specification, are meant to explain preferred embodiments of the present disclosure to those skilled in the art. Relevant FIGURES shown or described in the Detailed Description are as follows:

FIG. 1 shows a flow diagram of the different components of an example design.

FIG. 2 shows a pictorial representation of the top view of the data acquisition and transmission module.

FIG. 3 shows a pictorial representation of the bottom view of the data acquisition and transmission module.

FIG. 4 shows a pictorial representation of the side view of the data acquisition and transmission module.

FIG. 5 shows a pictorial representation of the left foot, front outside view of the wearable.

FIG. 6 shows a pictorial representation of the right foot, inside view of the wearable.

FIG. 7 shows a pictorial representation of the right foot, shoe view of the wearable, sensor, and module.

FIG. 8 shows a pictorial representation of the bottom view of the shoe view and sensor and sensor placement.

FIG. 9 illustrates a top view of an example ankle puck used for capturing sensor data.

FIG. 10 illustrates a bottom view of an example ankle puck used for capturing sensor data.

FIG. 11 illustrates a side view of an example ankle puck used for capturing sensor data.

FIG. 12 illustrates an example of a pair of ankle socks used for capturing sensor data.

FIG. 13 illustrates another example ankle sock used for capturing sensor data.

FIG. 14 illustrates an example graphical user interface used to display captured sensor data.

FIG. 15 is a schematic diagram of an example motion capture analysis device.

### DETAILED DESCRIPTION

The present disclosure provides a novel system and apparatus for wearable devices for humans and/or animals that obtains and records kinematic and kinetic data during real-time events, exercise, training, competition, and rehabilitation, for example, and that analyzes such data and movement and provides feedback, actionable information, and/or assessments to the wearer and/or to medical or training personnel about the wearer, as well as pertinent biomechanical data and assessments. The examples are useable in multiple applications, for both animals and humans, notably concerning sports, any type of training, rehabilitation, and the military, for example.

The present disclosure comprises a foot-ankle or body part wearable system comprising a wearable apparatus or device integrating one or more sensors, such as SRS sensors, into a wearable or compression garment or sock, for example, to capture kinematic and kinetic data during rigorous or non-rigorous training, testing, and task events in real-world environments. The system and device captures information about selected user joints, muscles, ligaments, bony landmarks, or a combination thereof, senses and monitors motion and movement of the joints, muscles, ligaments, bony landmarks, or a combination thereof of the user, and obtains real-time motion and movement parameter data. The disclosure further comprises one or more data acquisition and transmission modules, i.e., pucks, for providing power to the sensor and for receiving, transmitting, and storing real time, or near real time, motion and movement parameter data via a wired or wireless protocol for communicating with the sensor. The puck module has the ability to “store-it-forward” and transmit the raw data to a computer or computer-based device where a device app or application analyses and provides feedback based on the raw data. The puck module itself typically will not process the data, but can have the ability to optionally process the motion and movement data. Typically, the puck module at a minimum ensures proper data transmission with the appropriate timestamps. Further, the puck module of the disclosure has the ability to throttle and/or accelerate the data capture or refresh rate, i.e., the data collection time or rate, of the data from the sensors. Still further, the disclosure comprises a microprocessor-based data processing means or device for communicating with and receiving and analyzing the motion and movement parameter data from the puck, or data acquisition and transmission module, and for converting such data to motion and movement information and providing such information and characteristics about such motion and movement to the system user or subject being tested and/or an analyst. Such information and characteristics may include the intensity, duration, repetition, and the like, of such motion and movement.

Wearable is defined as an item to be worn or placed on a subject to be tested or analyzed, specifically as a flexible, rigid, or semi-rigid: sock, outerwear, underwear, compression wear, cover, sleeve, harness, band, or garment, or a combination thereof, for example, composed of polymeric or semi-polymeric material, fabric, non-fabric, substrate, a woven, non-woven, and/or knitted material and/or fabric, or a combination thereof. Flexible is defined typically, and as applied to wearables and to the sensors utilized by the disclosure, as stretchable, variable, bendable, twistable, compressible, pliable, pressable, malleable, and/or tension-able.

Data is or can be captured, saved or stored, and analyzed in real-time or near real-time using machine learning from modeling of the body part, or foot and ankle, movements and through the analysis of data collected in participant movement trials. Output and feedback from the device provides actionable, relevant information and/or assessments to the wearer, evaluator, medical staff, and/or trainer concerning various data parameters including, but not limited to, the level of risk associated with body part, joint, bony landmarks, or ankle movement and placement, the forces applied to the body part, joint, bony landmarks, or foot and ankle, symmetry across both of the wearer's paired joints or ankles, and additional biomechanical information, such as joint kinematics and inferred gait parameters

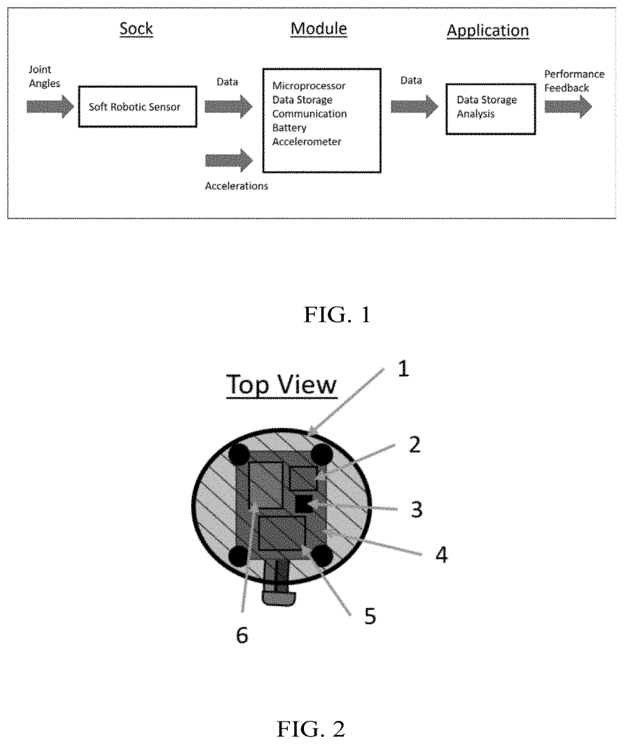

FIG. 1 shows a flow diagram of the different components of the design and shows how joint angle information is translated from movement into data using an SRS via a sock or body part wearable, that such information is stored and transmitted via a data acquisition and transmission module, and that relevant data is converted into human readable performance feedback via an application or microprocessor-based application.

FIGS. 2-8 show visualizations of a proposed prototypical embodiment and design for a wearable and a puck or data acquisition and transmission module, which in this embodiment attaches to shoelaces of a shoe and provides power to the sensor(s), provides redundant accelerometer readings, and transmits movement data via wired or wireless transmission to a microprocessor-based data processing means. These figures further show the prototypical wearable with preferred components and placements for this particular wearable.

The present disclosure utilizes key differentiators as compared to the current state of the art. The disclosure uses sensors such as SRSs to estimate (a) absolute joint angles at the foot and ankle and other relevant assessable bony landmarks, i.e., portions of the body where bones or joints are visually evident, and body parts, and (b) the specific movements of dorsiflexion, plantar flexion, inversion, eversion, abduction, and adduction, for ankles, for example. Current solutions must infer joint angles based on devices placed on limb segments. With inferred angles being the least precise, the use of relative angles can be valuable but have drawbacks based on their lack of consistency. Further, current wearable solutions do not use SRS sensors. SRS sensors are typically fabric-textile or silicone-textile, layered with liquid conductive material and generally identified as resistive, capacitive, or inductive. Advantages of SRS sensors include: (a) the ability to measure biomechanical strain without worry for occlusion errors typical in optical systems and elimination of drift in micro electromechanical device sensors (e.g. Inertial Measurement Units (IMUs)), (b) the realization of small changes in electromechanical readings during loading and unloading, and (c) the reduction of interference as observed by the wearer. In addition, SRS are inherently stretchable, which allows the sensors to cover arbitrarily-shaped human or animal joints. Focusing on SRS for movement capture mitigates issues commonly found in IMU sensors such as distortion and drift, magnetic field disturbance, and calibration challenges. Solutions for other joint wearables have begun to test SRS use, but true capability and functionality of such is unclear.

The disclosure brings subjects, athletes, rehabilitation specialists, different health care professionals, and trainers assessment information about the most injury prone parts of a human and animal body in high levels of training, rehabilitation, and athletic competition. The level of detail typically provided by current products has been limited to a laboratory environment and equipment such as motion capture and force plates. Users, athletes, and trainers may not have frequent access to this level of sophisticated equipment and performing training regimens within a laboratory may not be realistic or practical. Typically, little to no data feedback is available at this level of granularity. The disclosure brings an extremely precise and efficient level of feedback, particularly concerning absolute joint angle kinematic parameter data, from the laboratory into the actual environment where athletic training, rehabilitation, and real-life activities occur. Further, it allows complete transparency into data capture and calculations via algorithms and integration of the apparatus of the design into clothing and uniform requirements.

Alternative system and device embodiments and designs include an ankle or joint brace structure, integration into a shoe, sleeve, or harness, or simple elastic straps and/or Velcro-type straps to hold the SRS sensors in place, either directly on the body part or via the wearable. Moreover, alternate embodiments include multiple other specific sensor placement locations and sensors to monitor all six (6) ankle or other joint complex movements and forces or pressures of the foot on the ground, for example. The “puck” of the design is defined as and is a data acquisition and transmission module that provides power to the SRS sensor or other relevant type of stretch or liquid metal sensor and receives and transmits data values received from the sensors preferably via some form of wireless protocol (e.g., Bluetooth, Wi-Fi, and/or other form of IEEE 802.11 communications protocol or standard, for example) in which communication is provided to a receiving system, such as a mobile computing device. The puck module can accept, transmit, time-stamp, and store data in real-time from any type of robotic sensor type, resistive, capacitive, inductive, or a combination thereof, and can be placed anywhere on the individual being tested, analyzed, or monitored and is not limited to placement on a sock, shoe, and/or the ankle or joint complex region. The sensors can be placed in any number of multiple locations on or around the ankle, joint complex, or bony landmark to capture movement and angles, as well as on the bottom of the foot, within an insole, or in an optimal location near the body part to be analyzed and to record pressure and/or ground or other reaction forces and/or pressures. The disclosure provides specific optimal placement locations for the sensor(s). Further, such sensors can be integrated into fabrics/textiles/clothing, as depicted in FIGS. 2-8, or they can remain separate and anchored or attached to the ankle, specific joint or body part complex, or bony landmark using, for example, permanent or temporary adhesive materials. Alternatively, certain sensors can be both integrated into a wearable and the same or others attached to relevant body part complexes.

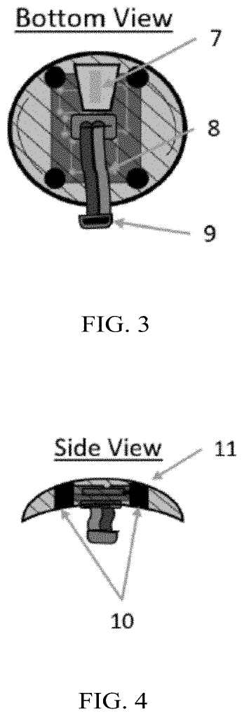

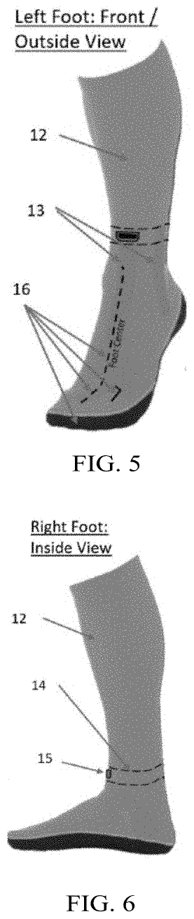

FIG. 2 shows a top-view visualization of a portion of the design for one particular body part application and embodiment, specifically a foot-ankle application, and more specifically the puck data acquisition and transmission module 1, which attaches to a user's shoelaces and provides power to the SRS while either sending data to the microprocessor means or performs data computations, via wired or wireless transmission thereto, and redundant accelerometer readings. FIGS. 2-8 show one embodiment of the manner in which the module 1 can be applied to or within clothing or a wearable to connect with sensors in a sock. The module 1 can also be fitted into a pouch on a sock, for example, as well as integration into the insole of a shoe. Attachment to the top of a shoe is but one of many such placement options. The module 1 comprises components housed within a ruggedized casing that covers and protects the module 1, and a bluetooth device 2, or similar communications device, such that the module 1 can connect to and provide communications through wireless protocols such as WiFi, BT, and/or cellular technology, for example, and house multiple antenna types, and whereby the module 1 provides wireless communication to the microprocessor means. Further, the module 1 comprises of an accelerometer 3, and/or other Intertial Measurement Unit (IMU) sensors, for example, that provides relevant motion and movement parameter data. Additionally, the module 1 comprises a board 4 that can be a printed circuit board, for example, for mounting the bluetooth 2, accelerometer 3, a lithium battery 5 which provides power to the module 1 and at least one sensor 13 (FIGS. 5, 7, and 8), and an optional module microprocessor or processor 6. FIG. 3 shows a bottom-view visualization of the module 1, specifically at least one shoelace clip 7, for a foot-ankle application, a cable 8 for power, charging, and/or data transmission to/from a sensor, and at least one sock connector 9 for connecting the board 4 to a sensor 13.

FIG. 4 shows a side-view visualization of the module 1, specifically at least one internal rigid support 10 and a curvature 11 to form fit the top of a shoe or the back of the leg or bottom of the foot via an insole, for example.

FIG. 5 shows, for an ankle sock embodiment, a left foot, front outside view of the wearable, specifically the wearable compression sock 12, the sensors 13, which can be Liquid Wire sensors, StretchSense sensors, or the like, and visual aids 16 for accurate, proper fit, and placement of the sock or wearable on the body part. FIG. 6 shows a right foot, inside view visualization of the wearable compression sock 12, specifically at least one compression band 14 and a module connector 15 to attach the module 1 and sensor 13 to the sock 12.

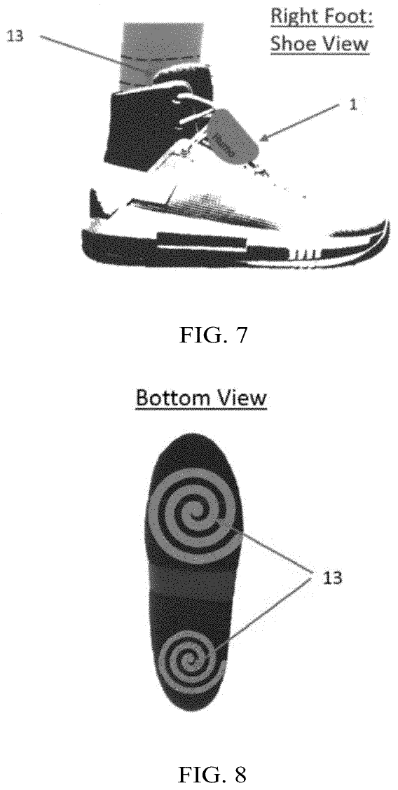

FIG. 7 shows a right foot, shoe view visualization of the design, specifically sensor 13 and module 1. FIG. 8 shows a bottom view visualization of the shoe view, specifically one embodiment of placement of sensors 13. The design further comprises a body part-specific, or ankle for example, microprocessor-based app or application that provides motion- and movement-specific feedback during and after training or assessment events for the wearer and/or trainer.

The sensors can be Liquid Wire sensors, StretchSense sensors, or any other soft robotic sensor that provides or demonstrates a linear relationship between movement and resistive, capacitive, inductive, or other electronic property output. Machine learning is used to translate sensor output(s) into movement analysis that can be interpreted as specific movements such as plantar flexion, dorsiflexion, inversion, eversion, abduction, and adduction for ankles, for example. The machine learning algorithm is specific to movement dimensions and demographics (e.g., subject individual human or animal height and weight) about a specific individual obtained via the software interface of the design. A computer-based and/or microprocessor-based system controls the system of the design. Further, a non-transitory computer-readable medium comprised of computer processor-based and/or microprocessor-based instructions utilize the system of the design to instruct a computer-based and/or microprocessor-based device to receive relevant data and provide relevant test or analysis subject individual information and assessment.

The examples can capture consistent and accurate data “from the ground up” for making health, training, and safety decisions about a wearers' ankles, joints, body parts, and other locations on the human or animal body where sensors are placed. Sensor placement locations typically include joints, knees, elbows, ligaments, feet, ankles, toes, legs, arms, hips, muscles, fingers, wrists, hands, head, neck, shoulders, any bony landmark, or a combination thereof, that provide or accommodate any animal or human body or body part motion or movement.

Additional information can be captured and learned when the device is placed on both feet, wrists, or compatible complementary body parts, joints, or bony landmarks. Such information includes insight into specific sensor placement, which is a key ingredient of the present disclosure, gait, gait assessment, leg asymmetry, and general movement performance, all of which factors are very specific to the individual or subject human or animal wearing the device. The wearable device and apparatus of the system provides the ability to accurately measure foot-ankle, or other joint, angles, heel and toe, or other body part or bony landmark, forces and pressures, either exerting or receiving, allows combining joint angle and force/pressure measurements into machine learning parameters that estimate injury risk, and allows trainers and analysts to better assess and monitor subjects.

The design can be used on all joints of the human and/or animal body and is not limited to the ankle complex. For example, a wrist design includes liquid metal sensors integrated into a glove and captures the complex movements of the wrist, as well as force (i.e., grip strength) that occurs between the thumb and/or other multiple fingers. Additionally, motion and movement of finger, hand, wrist, elbow, shoulder, hip, knee, foot, and other similar body parts and bony landmarks to be tested, analyzed, and assessed can be integrated into the scheme. Other joints of the human and animal body can likewise have motion and movement captured and analyzed using alternative embodiments or variations of the design, depending on specific sensor placement and machine learning algorithm(s) for specific movement models.

While the design is applicable to athletics, the capabilities of the design benefit both athletic and non-athletic individuals and animals including the industrial, military, and sports athlete, as well as any subject in recovery or rehabilitation from an injury or in training to prevent an injury. The design provides a supplement to or replacement of expensive orthopedic gait assessment equipment, for example, to make assessing and quantifying recovery more accessible, particularly when such movement assessment is otherwise inaccessible. For example, goniometer technology is typically a simple single plane, single dimension measurement process for measuring range of motion around a body joint, while three-dimensional motion capture technology for such measurement is lab-based and expensive. On the other hand, the present design is highly accurate, efficient, inexpensive, multi-dimensional in scope, and both lab and field useable and compatible.

The design is comprised of a foot-ankle, or other joint or body part or bony landmark, wearable integrating a stretch-type sensor, such as an SRS, into a wearable device, clothing, or sock or compression sock, or similar clothing material, to capture kinematic and kinetic data during exercise, rigorous training, competition, and/or task events in real-world and/or rehabilitation environments. Relevant motion and movement data is captured, stored, and analyzed in real-time or near real-time using machine learning from modeling of foot and ankle movements, or relevant joint or body part movements, and through analysis of data collected in participant movement trials. Output and feedback from the device provides actionable information to the wearer and/or trainer about the level of risk associated with foot, ankle, joint, or body part movement and placement, the forces applied to the foot, ankle, joint, and/or body part, symmetry across a wearer's relevant body measurement points, and additional biomechanical information on movement patterns, such as gait, distance, and jumping and dynamic compound movements, absolute joint angle, asymmetry, force, temperature, pressure, pulse rate, joint movement data including flexion, extension, hyperextension, circumduction, supination, pronation, rotation, protraction, retraction, elevation, depression, opposition, plantar flexion, dorsiflexion, inversion, eversion, abduction, and adduction, grip strength, joint strength, or a combination thereof, for example. The design provides consistent, reliable, and accurate real-time data “from the ground up” for making health and safety decisions about a wearer's relevant joints and other measurable body portion(s) of the human or animal body to be assessed and analyzed. The design provides training and performance and movement assessment via wearables to capture joint movement and relevant real-time biomechanical parameters for analysis. The design provides optimized specific sensor number and placement, gait assessment (for ankles and feet) validation against motion capture, jumping, running, and other similar dynamic compound movement assessment, for example, machine learning algorithms specific to ankle and joint complex movements, and sensor anchoring designs and textile integration.

All parameters presented herein including, but not limited to, sizes, dimensions, times, temperatures, pressures, amounts, distances, quantities, ratios, weights, volumes, percentages, and/or similar features and data and the like, for example, represent approximate values and can vary with the possible embodiments described and those not necessarily described but encompassed by the design. Further, references to ‘a’ or ‘an’ concerning any particular item, component, material, or product is defined as at least one and could be more than one.

The following is a detailed example implementation of the aspects described above. For example, the wearable devices described herein can be configured to detect various health conditions. As a further example, sensors attached to a sock may forward data to an application operating on a computer, tablet, phone or other electronic device. The application may then indicate the detected data and/or potential health issues based on such data. As an example, an light emitting diode (LED) can be included in a sock and positioned adjacent to, and/or in contact with, an ankle to allow for pulse detection, which can be used to indicate a wide range of health issues.

In another example, the sensors in a sock may measure a user's gait when walking and store such data. The application may compare past profiles of the user's gait to a present profile of the user's gait to determine changes in gait over time. As a specific example, such changes in gait can be used to for detect that a user has suffered from a stroke. The aspects may also perform other types of gait analysis. For example, the application may analyze the data to detect and indicate: an abnormal gait, a user's gait type (e.g., flat footed), slip and/or falls, and/or perform other general gait parameter estimations.

In yet another example, stretch and/or strain-based SRSs can be include in the sock and can be positioned around a user's joints, foot, calf, etc. The SRSs can be used to quantify changes in the circumference (e.g. diameter) of the user's joints, foot, calf, etc. and send such measurements to the application. The application can then use the changes in circumference to indicate and/or quantify edema or other swelling related issues in the user's foot.

Several examples of foot and ankle pressure related features are listed below. The sensors involved in each example may forward the relevant sensor data to an application for display to an operator and/or for analysis by an application, artificial intelligence, or other computing system. In the artificial intelligence context, such data can be forwarded to an artificial intelligence (AI) and/or machine learning (ML) network for training. Such a network may include feedforward neural network, group method of data handling (GMDH) neural network, autoencoder network, probabilistic neural network (PNN), time delay neural network (TDNN), convolutional neural network (CNN), deep stacking network (DSN), tensor deep stacking network, or combinations thereof. In addition, a deep network that utilizes inputs and creates semantic statements can be utilized to provide human-understandable information, e.g., “The right leg is being highly favored during jumping indicating a potential injury risk.” A neural network can be provided with sensor results and associated diagnosis information as data sets for use as training data during a training phase. The AI can include a network of decision nodes connected according to various weights. The AI can perform an analysis of training sensor data along the decision nodes and compare the results to the associated training diagnosis information. The AI can then increase or decrease weights depending on the accuracy of the analysis of the training sensor data when compared to the associated training diagnosis information. Once the AI is trained on a large data set (e.g., thousands or more data points), the AI can use the sensor data to (a) aid in diagnosis of medical conditions, (b) estimate gait or other limb movement parameters, (c) provide trainer feedback to improve performance, and (d) provide information for trainers or medical staff over time for specified movements.

For example, sensors can capture foot-ankle complex planter flexion in a sagittal plane. This can be accomplished using a stretch and/or strain-based SRS located at a bony landmark location of an anterior aspect of the foot and ankle along a midline of an ankle joint axis extending proximally to a distal tibia and fibula and distally to a talus and a 3rd metatarsal.

As another example, sensors can capture foot-ankle complex dorsiflexion in the sagittal plane. This can be using a stretch and/or strain-based SRS located at a bony landmark location of a posterior aspect of a foot and ankle along a midline of a calcaneum distally and a midline of an Achilles tendon.

As another example, sensors can capture foot-ankle complex inversion in a frontal plane. This can be accomplished using a stretch and/or strain-based SRS located at a bony landmark location of a lateral aspect of a foot and ankle along a midline of a lateral malleolus of a fibula proximally, and a talus, calcaneum, and cuboid distally.

As another example, sensors can capture foot-ankle complex eversion in a frontal plane. This can be accomplished using a stretch and/or strain-based SRS located at a bony landmark location of a medial aspect of a foot and ankle along a midline of a medial malleolus of a tibia proximally, and a talus and calcaneum distally.

As another example, sensors can capture foot-ankle complex triplanar movement of pronation. This includes movement in three planes and may specifically include movements of foot abduction in a transverse plane, dorsiflexion in a sagittal plane, and eversion in a frontal plane. This can be accomplished with combinations of the sensors (e.g., stretch and/or strain-based SRSs) with dorsiflexion, plantar flexion, inversion, and eversion sensor placements.

As another example, sensors can capture foot-ankle complex triplanar movement of supination. This includes movements of foot adduction in a transverse plane, plantar flexion in a sagittal plane, and inversion in a frontal plane. This can be accomplished with combinations of the sensor (e.g., stretch and/or strain-based SRSs) with dorsiflexion, plantar flexion, inversion, and eversion sensor placements.

As another example, sensors can capture a combination of (a) a comprehensive multiplanar foot-ankle movement, (b) foot-ground pressure, and (c) ground reaction forces (vertical and sheer) from the foot. Such data can be forwarded to a computing system employing artificial intelligence and/or machine learning for analysis, detection, and/or diagnosis of medical conditions. This can be accomplished by (a) stretch/strain-based soft robotic sensors located at bony landmarks to detect the comprehensive multiplanar foot-ankle movement, (b) pressure-based soft robotic sensors to detect foot-ground pressure where such sensors are located in a combination of one or more (e.g., all) of the following locations: the first metatarsal, fifth metatarsal, calcaneus, and big toe (hallux), and (c) a combination of pressure-based soft robotic sensors and/or IMUs to detect ground reaction forces on the foot. Such data can be forwarded to an AI/ML for neural network training.

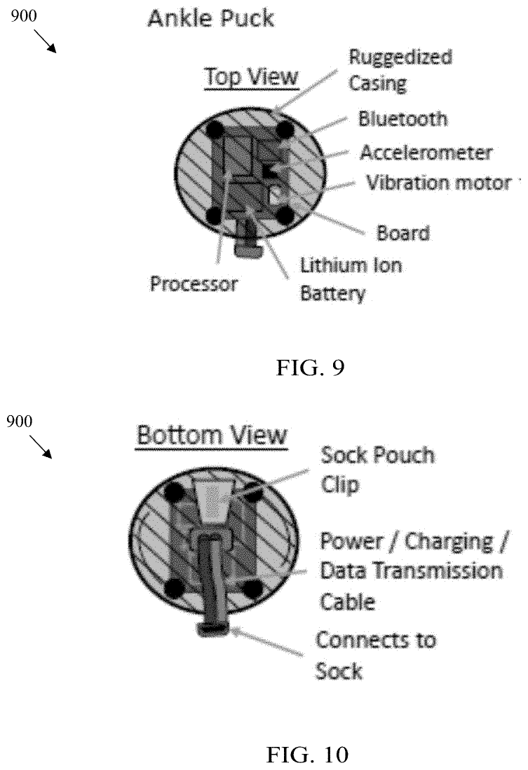

FIG. 9 illustrates a top view of an example ankle puck 900 used for capturing sensor data. The ankle puck 900 is configured to be included in a pouch in the sock and for receiving a processing and/or preprocessing sensor data. The ankle puck 900 contains a board for containing various circuitry. The board is contained within a ruggedized casing, which protects circuitry when the ankle puck 900 is in use. The board also contains a battery (e.g. lithium ion) for powering the board and other components. The board also contains a wireless interface, such as a bluetooth interface, for communicating with a computer, table, smartphone, or other electronic computing device. The wireless interface is used to forward sensor data to the computing device for further analysis and/or display. The board may also contain an accelerometer, which is a sensor used to measure motion, for example to determine gait. The board may also contain a vibration motor, which can be used in conjunction with other sensor to measure responses in a user's leg to the vibration.

FIG. 10 illustrates a bottom view of an example ankle puck 900 used for capturing sensor data. As shown in FIG. 10, the ankle puck 900 contains a sock pouch clip used for securing the ankle puck 900 within a sock pouch during use. The board contains ports for receiving sensor data and a processor for processing the sensor data. For example, the board can be connected to a removable power cable for charging via a port. Further, the board can be connected to a removable data transmission cable, which can be connected to sensors in the sock.

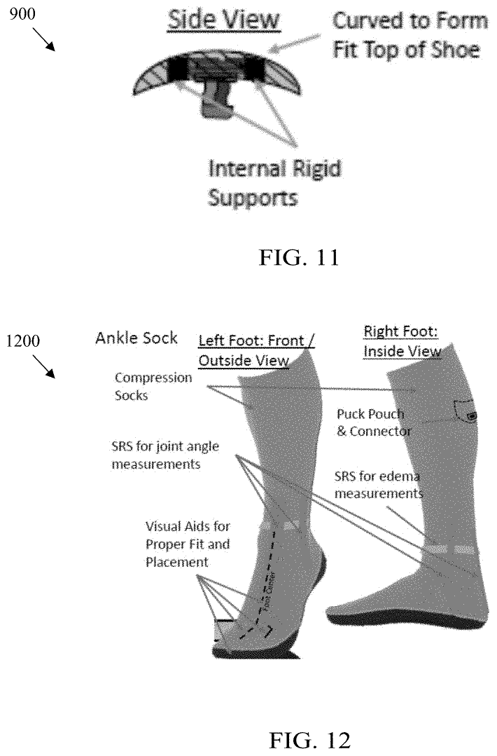

FIG. 11 illustrates a side view of an example ankle puck 900 used for capturing sensor data. As shown, the casing of the ankle puck 900 may employ a curved form factor. This allows the casing to fit well within the sock pouch or within a shoe, depending on the example. The ankle puck 900 may further contain internal rigid supports connecting the board to the casing. The supports maintain the board in place and prevent movement of circuitry when the ankle puck 900 is in use.

FIG. 12 illustrates an example of a pair of ankle socks 1200 used for capturing sensor data. An ankle sock may contain a puck pouch and connector for receiving the ankle puck 900. The puck pouch retains the ankle puck 900 while the user moves within the ankle sock. The connector is used to connect to the data transmission cable. Additional wires can be used to connect the connector in the puck pouch to the various sensors in the ankle sock. In some examples, two ankle pucks 900 can be used and placed in pouches in each of a left sock and a right sock. This avoids a need to provide wires between the left sock and right sock. The pair of ankle socks 1200 may each be configured as compression socks to ensure sensors are pressed firmly against a user's legs, feet, etc. for accurate measurements. The pair of ankle socks 1200 may each be labeled with visual aids to allow the user to correctly put on each sock. The visual aids help ensure the sensors are placed correctly against the corresponding body features. As discussed above, the ankle socks 1200 may contain many combinations of sensors. In this example, an SRS is positioned around the leg above the ankle and configured to measure for signs of edema and other swelling related issues. The SRS may also be configured to measure for joint angle movements.

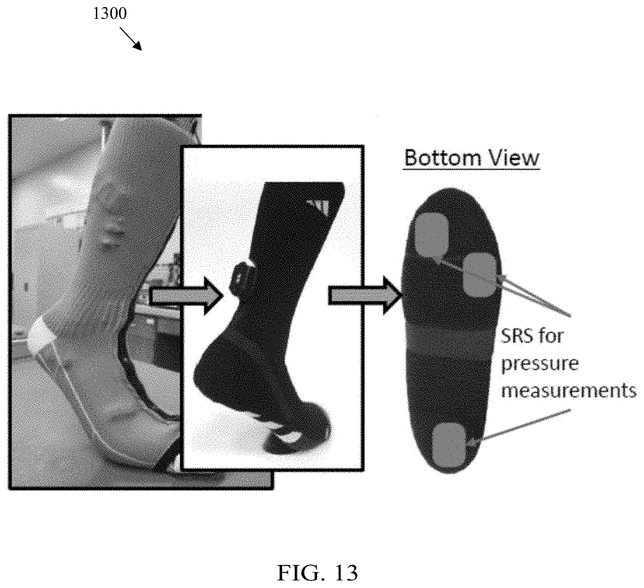

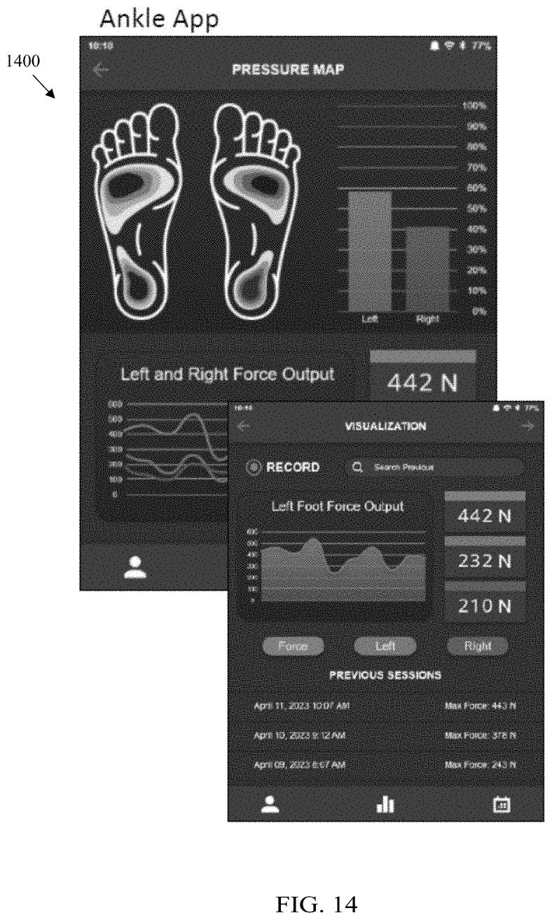

FIG. 13 illustrates another example ankle sock 1300 used for capturing sensor data. As shown, the ankle sock 1300 can contain various sensors around a user's leg, foot, and ankle. Further, sensors can be placed on the bottom of the sock to abut the bottom of a user's foot in various locations. The sensors can be configured as SRS pressure sensor and can measure vertical force applied by the user when walking.

FIG. 14 illustrates an example graphical user interface 1400 used to display captured sensor data. The graphical user interface 1400 can be generated by a computer, tablet, phone, or other computing device, such as a motion capture analysis device 1500 as described below. For example, the ankle puck 900 can receive sensor data from the ankle socks 1200 and/or the ankle sock 1300. The ankle puck 900 can then connect to the computing device via a wireless interface and communicate the sensor data to the computing device. The computing device can then analyze the data, apply AI to the data, and/or display the data and related analysis results on the graphical user interface 1400. In the example shown, the pressure sensors on the bottom of a sock have measured force (e.g., ground pressure) acting upon the bottom of a user's feet. The graphical user interface 1400 displays a heat map showing position of the force as well as recorded force over time. This data can be used by an operator to diagnose issues related to gait, gait type, gait changes, or other gait related issues.

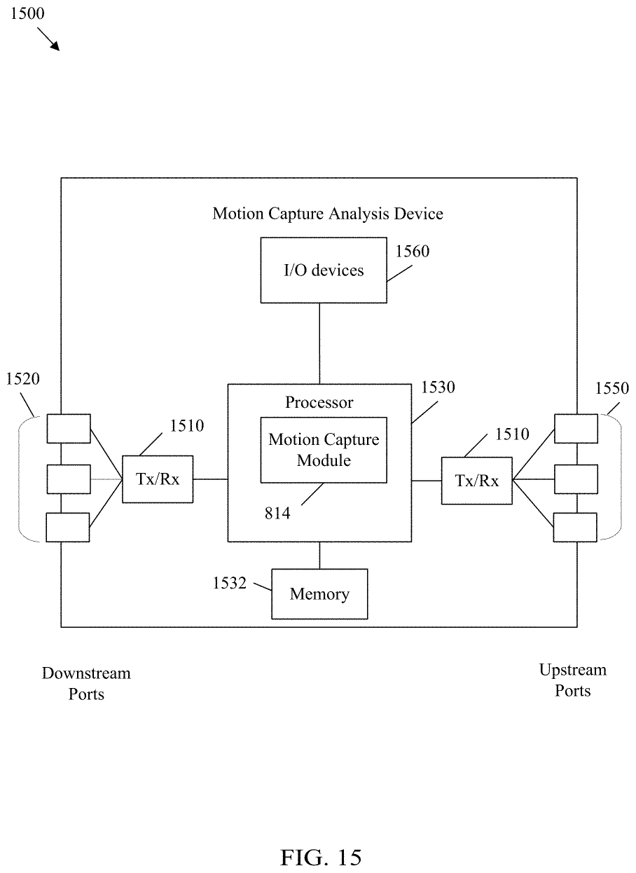

FIG. 15 is a schematic diagram of an example motion capture analysis device 1500. The motion capture analysis device 1500 is suitable for implementing the disclosed examples/embodiments as described herein. The motion capture analysis device 1500 comprises downstream ports 1520, upstream ports 1550, and/or transceiver units (Tx/Rx) 1510, including transmitters and/or receivers for communicating data upstream and/or downstream over a network. The motion capture analysis device 1500 also includes a processor 1530 including a logic unit and/or central processing unit (CPU) to process the data and a memory 1532 for storing the data. The motion capture analysis device 1500 may also comprise electrical, optical-to-electrical (OE) components, electrical-to-optical (EO) components, and/or wireless communication components coupled to the upstream ports 1550 and/or downstream ports 1520 for communication of data via electrical, optical, or wireless communication networks. The motion capture analysis device 1500 may also include input and/or output (I/O) devices 1560 for communicating data to and from a user. The I/O devices 1560 may include output devices such as a display for displaying video data, speakers for outputting audio data, etc. The I/O devices 1560 may also include input devices, such as a keyboard, mouse, trackball, etc., and/or corresponding interfaces for interacting with such output devices.

The processor 1530 is implemented by hardware and software. The processor 1530 may be implemented as one or more CPU chips, cores (e.g., as a multi-core processor), field-programmable gate arrays (FPGAs), application specific integrated circuits (ASICs), and digital signal processors (DSPs). The processor 1530 is in communication with the downstream ports 1520, Tx/Rx 1510, upstream ports 1550, and memory 1532. The processor 1530 comprises a motion capture module 1514. The motion capture module 1514 implements the disclosed embodiments described above, such as implementing AI/ML, receiving sensor data, analyzing sensor data, displaying sensor data, providing diagnosis or other sensor data analysis, or combinations thereof. The motion capture module 1514 may also implement any other method/mechanism described herein. As such, the motion capture module 1514 causes the motion capture analysis device 1500 to provide additional functionality by analyzing and displaying motion capture or other sensor data. As such, the motion capture module 1514 improves the functionality of the motion capture analysis device 1500 as well as addresses problems that are specific to the health diagnostics arts. Further, the motion capture module 1514 effects a transformation of the motion capture analysis device 1500 to a different state. Alternatively, the motion capture module 1514 can be implemented as instructions stored in the memory 1532 and executed by the processor 1530 (e.g., as a computer program product stored on a non-transitory medium).

The memory 1532 comprises one or more memory types such as disks, tape drives, solid-state drives, read only memory (ROM), random access memory (RAM), flash memory, ternary content-addressable memory (TCAM), static random-access memory (SRAM), etc. The memory 1532 may be used as an over-flow data storage device, to store programs when such programs are selected for execution, and to store instructions and data that are read during program execution.

The above detailed description is presented to enable any person skilled in the art to make and use the design. Specific details have been revealed to provide a comprehensive understanding of the present design and are used for explanation of the information provided. These specific details, however, are not required to practice the design, as is apparent to one skilled in the art. Descriptions of specific applications, analyses, materials, components, dimensions, and calculations are meant to serve only as representative examples. Various modifications to the preferred embodiments may be readily apparent to one skilled in the art, and the general principles defined herein may be applicable to other embodiments and applications while still remaining within the scope of the disclosure. There is no intention for the present disclosure to be limited to the embodiments shown and the design is to be accorded the widest possible scope consistent with the principles and features disclosed herein.

While various embodiments of the present disclosure have been described above, it should be understood that they have been presented by way of example and not limitation. It will be apparent to persons skilled in the relevant art(s) that various changes in form and detail can be made therein without departing from the spirit and scope of the present disclosure. In fact, after reading the above description, it will be apparent to one skilled in the relevant art(s) how to implement the design in alternative embodiments. This disclosure has described the preferred embodiments of the disclosure, but it should be understood that the broadest scope of the disclosure includes such modifications as additional or different methods and materials. Many other advantages of the disclosure will be apparent to those skilled in the art from the above descriptions and the subsequent claims. Thus, the present disclosure should not be limited by any of the above-described exemplary embodiments.

The processes, devices, products, apparatus and designs, systems, configurations, methods and/or compositions of the present disclosure are often best practiced by empirically determining the appropriate values of the operating parameters or by conducting simulations to arrive at best design for a given application. Accordingly, all suitable modifications, combinations, and equivalents should be considered as falling within the spirit and scope of the disclosure.

What is claimed is:

1. A sock comprising:
a plurality of soft robotic sensors (SRSs) integrated into a fabric, wherein the SRSs are configured to sense and monitor motion and movement of bony landmarks of a wearer in a plurality of planes, and obtain real-time motion and movement parameter data; and
a puck electrically coupled to the SRSs and configured to:
provide power to the SRSs,
receive sensor data from the SRSs,
store data until wireless transmission can occur, and
wirelessly transmit the sensor data to a computing device.

2. The sock of claim 1, wherein the SRSs comprise a plantar flexion SRS configured to abut a dorsal surface a foot and to measure downward movement of the foot, and wherein the SRSs further comprise an eversion SRS configured to be abut a medial side of an ankle of the foot and to measure movement of a sole of the foot away from a midline of a body for data capture in the plurality of planes.

3. The sock of claim 1, wherein the SRSs comprise an inversion SRS configured to abut on a lateral side of an ankle of a foot and to measure movement of a sole of the foot toward a midline of a body, and wherein the SRSs further comprise a dorsiflexion SRS configured to abut a heel of the foot and to measure upward movement of the foot for data capture in the plurality of planes.

4. The sock of claim 1, wherein the SRSs comprise an SRS configured to abut a bony landmark location of an anterior aspect of a foot and an ankle along a midline of an ankle joint axis extending proximally to a distal tibia and fibula and distally to a talus and a 3rd metatarsal, and further configured to capture foot-ankle complex planter flexion in a sagittal plane.

5. The sock of claim 1, wherein the SRSs comprise an SRS configured to abut a bony landmark location of a posterior aspect of a foot and ankle along a midline of a calcaneum distally and a midline of an Achilles tendon, and further configured to capture foot-ankle complex dorsiflexion in a sagittal plane.

6. The sock of claim 1, wherein the SRSs comprise an SRS configured to abut a bony landmark location of a lateral aspect of a foot and ankle along a midline of a lateral malleolus of a fibula proximally, and a talus, calcaneum, and cuboid distally, and further configured to capture foot-ankle complex inversion in a frontal plane.

7. The sock of claim 1, wherein the SRSs comprise an SRS configured to abut a bony landmark location of a medial aspect of a foot and ankle along a midline of a medial malleolus of a tibia proximally, and a talus and calcaneum distally, and further configured to capture foot-ankle complex eversion in a frontal plane.

8. The sock of claim 1, wherein the SRSs are further configured with dorsiflexion, plantar flexion, inversion, and eversion sensor placements, and further configured to capture foot-ankle complex triplanar movement of pronation including foot abduction in a transverse plane, dorsiflexion in a sagittal plane, and eversion in a frontal plane.

9. The sock of claim 1, wherein the SRSs are further configured with dorsiflexion, plantar flexion, inversion, and eversion sensor placements and further configured to capture foot-ankle complex triplanar movement of supination including movements of foot adduction in a transverse plane, plantar flexion in a sagittal plane, and inversion in a frontal plane.

10. The sock of claim 1, wherein the SRSs comprise:
one or more stretch or strain SRS configured to be located at bony landmarks and configured to detect a comprehensive multiplanar foot-ankle movement,
one or more pressure-based SRS configured to be located at a first metatarsal, a fifth metatarsal, a calcaneus, a big toe, or combinations thereof and configured to detect foot-ground pressure, and
one or more pressure-based SRS configured to detect ground reaction forces on a foot.

11. The sock of claim 1, wherein the sensor data is wirelessly transmitted to the computing device to train a neural network to diagnose medical conditions.

12. A motion capture analysis device comprising:
a receiver configured to receive sensor data from a plurality of SRS via a puck, the sensor data including real-time motion and movement parameter data related to motion and movement of bony landmarks, in a plurality of planes, of a wearer of a sock containing the plurality of SRS; and
perform machine learning based on the sensor data to indicate risk associated with ankle movement.

13. The motion capture analysis device of claim 12, wherein the sensor data is associated with:
a plantar flexion SRS configured to abut a dorsal surface a foot and to measure downward movement of the foot, and
an eversion SRS configured to be abut a medial side of an ankle of the foot and to measure movement of a sole of the foot away from a midline of a body for data capture in the plurality of planes.

14. The motion capture analysis device of claim 12, wherein the sensor data is associated with:
an inversion SRS configured to abut on a lateral side of an ankle of a foot and to measure movement of a sole of the foot toward a midline of a body, and
a dorsiflexion SRS configured to abut a heel of the foot and to measure upward movement of the foot for data capture in the plurality of planes.

15. The motion capture analysis device of claim 12, wherein the sensor data is associated with an SRS configured to abut a bony landmark location of an anterior aspect of a foot and an ankle along a midline of an ankle joint axis extending proximally to a distal tibia and fibula and distally to a talus and a 3rd metatarsal, and further configured to capture foot-ankle complex planter flexion in a sagittal plane.

16. The motion capture analysis device of claim 12, wherein the sensor data is associated with an SRS configured to abut a bony landmark location of a posterior aspect of a foot and ankle along a midline of a calcaneum distally and a midline of an Achilles tendon, and further configured to capture foot-ankle complex dorsiflexion in a sagittal plane.

17. The motion capture analysis device of claim 12, wherein the sensor data is associated with an SRS configured to abut a bony landmark location of a lateral aspect of a foot and ankle along a midline of a lateral malleolus of a fibula proximally, and a talus, calcaneum, and cuboid distally, and further configured to capture foot-ankle complex inversion in a frontal plane.

18. The motion capture analysis device of claim 12, wherein the sensor data is associated with an SRS configured to abut a bony landmark location of a medial aspect of a foot and ankle along a midline of a medial malleolus of a tibia proximally, and a talus and calcaneum distally, and further configured to capture foot-ankle complex eversion in a frontal plane.

19. The motion capture analysis device of claim 12, wherein the sensor data is associated with SRS employing dorsiflexion, plantar flexion, inversion, and eversion sensor placements and configured to capture foot-ankle complex triplanar movement of supination including movements of foot adduction in a transverse plane, plantar flexion in a sagittal plane, and inversion in a frontal plane.

20. The motion capture analysis device of claim 12, wherein the sensor data is associated with:
one or more stretch or strain SRS configured to be located at bony landmarks and configured to detect a comprehensive multiplanar foot-ankle movement,
one or more pressure-based SRS configured to be located at a first metatarsal, a fifth metatarsal, a calcaneus, a big toe, or combinations thereof and configured to detect foot-ground pressure, and
one or more pressure-based SRS configured to detect ground reaction forces on a foot.
